(19) United States
(12) Patent Application Publication
(10) Pub. No.: US 2023/0398060 A1
(43) Pub. Date: Dec. 14, 2023

(54) TOPICAL COMPOSITIONS AND METHODS FOR TREATING PAIN

(71) Applicant: MIG USA, LLC, High Point, NC (US)

(72) Inventors: Adnan Mjalli, High Point, NC (US); Bill Hitchcock, High Point, NC (US)

(21) Appl. No.: 18/033,104

(22) PCT Filed: May 19, 2021
(86) PCT No.: PCT/US21/33174

§ 371 Date: Apr. 21, 2023

Related U.S. Application Data
(60) Provisional application No. 63/027,040, filed on May 19, 2020.
(60) Provisional application No. 63/027,125, filed on May 19, 2020.
(60) Provisional application No. 63/027,135, filed on May 19, 2020.
(60) Provisional application No. 63/027,149, filed on May 19, 2020.

(51) Int. Cl. A61K 9/00; A61K 9/06; A61K 47/36; A61K 31/192; A61K 31/10; A61K 31/616; A61K 31/00; A61K 31/137; A61K 31/606; A61K 31/196; A61P 29/00

(52) U.S. Cl. CPC A61K 9/0019; A61K 9/06; A61K 47/36; A61K 31/192; A61K 31/10; A61K 31/616; A61K 31/658; A61K 31/137; A61K 31/606; A61K 31/196; A61P 29/00

(57) ABSTRACT

Disclosed are compositions and methods for the treatment of pain and/or inflammation. In certain embodiments, the disclosed compositions and methods have anti-inflammatory activity. In some embodiments, the disclosed compositions and methods have analgesic activity. The present disclosure provides that two non-steroidal anti-inflammatory agents, as for example, 2-(4-isobutylphenyl)propanoic acid (ibuprofen), acetylsalicylic acid (aspirin), a muscle relaxant, and cannabidiol (CBD) can be used in combination in a topical formulation as an effective analgesic and/or anti-inflammatory agent.

### RELATED APPLICATIONS

This application claims priority to U.S. provisional patent application No. 63/027,040 filed on May 19, 2020, U.S. provisional patent application No. 63/027,125 filed on May 19, 2020, U.S. provisional patent application No. 63/027,135 filed on May 19, 2020, and U.S. provisional patent application No. 63/027,149 filed on May 19, 2020. The disclosures of U.S. provisional patent application Nos. 63/027,040, 63/027,125, 63/027135, 63/027149 are incorporated by reference in their entirety herein.

### FIELD OF THE INVENTION

The present disclosure relates to topical compositions for the treatment of inflammation and pain and methods for the treatment of inflammation and pain using topical compositions.

### BACKGROUND

Pain is a localized or generalized unpleasant sensation and can be either chronic or acute. Treatment of pain can be challenging due in part to the adverse side effects associated with currently available therapeutics. One of the most commonly used therapeutics for the treatment of pain are nonsteroidal anti-inflammatory drugs (NSAIDs). NSAIDs work by blocking cyclooxygenase (COX) enzymes responsible for producing prostaglandins, which are known to promote inflammation and pain. NSAIDs can be used for a variety of purposes, including pain relief, inflammation reduction, fever reduction, and prevention of blood clots. Two of the most common over-the-counter NSAIDs are 2-(4-isobutylphenyl)propanoic acid (ibuprofen) and acetylsalicylic acid (aspirin).

Both ibuprofen and aspirin are most commonly administered orally. However, oral consumption of NSAIDs has been associated with several cardiovascular and gastrointestinal complications, including nausea, vomiting, indigestion, gastric ulceration, and diarrhea. As such, oral NSAIDs may be contraindicated for those with a history of cardiovascular or gastrointestinal issues. Topical administration of NSAIDs has been considered; however, efficient transdermal delivery of therapeutically active doses has proven challenging. Further, when higher doses of NSAIDs are administered topically, the incidence of adverse dermal reactions has been shown to increase.

A variety of other compounds have been demonstrated to be effective in treating pain and inflammation. One such compound is cannabidiol (CBD). CBD oil, made from cannabis has been used for the treatment of inflammation and both acute and chronic pain.

Also, muscle relaxants may be used to treat pain associated with muscle spasms from musculoskeletal conditions. One such muscle relaxant is cyclobenzaprine. Like ibuprofen and aspirin, cyclobenzaprine is commonly administered orally. Common side effects include headache, drowsiness, and dizziness. Due to the sedative effects of cyclobenzaprine, oral administration may not always be suitable.

Thus, there is a need to develop new formulations of analgesic compounds as well as therapeutic carrier systems that can be topically applied for the treatment of pain and inflammation. Topical administration of NSAIDs, a muscle relaxant such as cyclobenzaprine, and CBD allows for targeted site action while allowing plasma levels of the active ingredients to remain low, and thus, limiting potentially harmful side effects. The delivery of ibuprofen, cyclobenzaprine, aspirin, and CBD in a chitosan-based gel carrier system present a promising strategy.

### SUMMARY OF THE DISCLOSURE

In a first aspect, disclosed herein is a composition formulated for topical application comprising: a therapeutically effective amount of one or more analgesic active ingredients and a carrier gel comprising chitosan. In some embodiments the carrier gel comprises chitosan powder, a gelling catalyst, and a solvent. In some embodiments, the active ingredient comprises a nonsteroidal anti-inflammatory drug (NSAID). In further embodiments, the composition further comprises one or more additional NSAIDs comprising at least one of acetylsalicylic acid (aspirin), methyl salicylate, sodium salicylate, and trolamine salicylate. In another embodiment, the composition further comprises additional therapeutics. For example, the composition may also comprise a muscle relaxant (e.g, cyclobenzaprine) and/or cannabidiol (CBD).

In a second aspect, disclosed herein is a method for preventing and/or treating and/or reducing pain and/or inflammation in a subject comprising topically administering to the subject any one of the compositions disclosed herein.

The present disclosure provides compositions and methods for the treatment of pain and/or inflammation. In certain embodiments, the disclosed compositions and methods have anti-inflammatory activity. In some embodiments, the disclosed compositions and methods have analgesic activity.

### DETAILED DESCRIPTION

The presently disclosed subject matter now will be described more fully hereinafter with reference to the accompanying description, in which some, but not all embodiments of the presently disclosed subject matter are shown. The disclosed subject matter can be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the disclosed subject matter set forth herein will come to mind to one skilled in the art to which the disclosed subject matter pertains having the benefit of the teachings presented in the description. Therefore, it is to be understood that the disclosed subject matter is not to be limited to the specific embodiments disclosed herein and that modifications and other embodiments are intended to be included within the scope of the appended claims. Although specific terms are employed herein, they are used in a generic and descriptive sense only and not for purposes of limitation.

### Definitions

While the following terms are believed to be well understood by one of ordinary skill in the art, the following definitions are set forth to facilitate explanation of the presently disclosed subject matter. Other definitions are found throughout the specification. Unless otherwise defined, all technical and scientific terms used herein have the same meaning as commonly understood by one of ordinary skill in the art to which this presently described subject matter belongs.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

The terms “a”, “an”, and “the” refer to “one or more” when used in this application, including the claims. Thus, for example, reference to “a cell” includes a plurality of such cells, unless the context clearly is to the contrary (e.g., a plurality of cells), and so forth.

The term “or”” is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “about” is used to indicate that a value includes the inherent variation of error for the device, the method being employed to determine the value, or the variation that exists among sample.

The term “bioadhesive” refers to natural polymeric materials that act as adhesives.

The term “buffer solution” is used to indicate a solution that resists a change in pH hen hydrogen ions (H⁺) or hydroxide ions (OH⁻) are added. A buffered solution may be produced by mixing a weak acid with its conjugate base. The buffer solution may be added to water to create “buffered water.”

The term “gelling catalyst” is used to refer to any substance that increase the rate of reaction of converting a sol into a gel (i.e., gelling).

The term “gel” is used to refer to a sol in which the solid particles are meshed such that a rigid or semi-rigid mixture results.

The term “solvent” is used to refer to any substance, typically a liquid, in which other substances dissolve.

Topical Composition

In one aspect, provided herein is a composition formulated for topical application comprising a therapeutically effective amount of one or more analgesic active ingredients and a carrier gel comprising chitosan. The carrier gel may, in certain embodiments, comprise at least one of a chitosan powder, a gelling catalyst, and/or a solvent.

In some embodiments, the carrier gel for the active ingredients comprises a chitosan. Chitosan has been demonstrated to have several uses including as an anti-cancer agent, a wound healing agent, and an antimicrobial agent. Alsarra (2009) International Journal of Biological Macromolecules 45: 6-21. Chitosan-based gels are ideal carriers for topically delivering therapeutic agents due to their low toxicity, biocompatibility, and non-immunogenic properties.

Chitosan is a deacetylated derivative of chitin that is made by treating the chitin found in the shells of shellfish with an alkaline substance. Chitosans are understood to be a family of binary heteropolysaccharides composed of β-1→4 linked 2-acetamido-2-deoxy-β-d-glucopyranose (GlcNAc, the “acetylated”, i.e., the A unit) and 2-amino-2-deoxy-β-d-glucopyranose (GlcNH₂, the “deacetylated”, i.e., the D unit) residues, present in different relative proportion and sequence along the chain. Sacco et al. (2018) Gels 4(3):67. The degree of deacetylation of chitosan provides the molar percentage of glucosamine monomeric units and varies from 0 (chitin) to 100 (fully deacetylated chitin). The amount of deacetylation affects the ability of chitosan to undergo the transition to a gel as well as the overall stability of the gel. In certain instances, the carrier gel comprises chitosan. In some embodiments, the chitosan gel is made using deacetylated chitosan powder. In some embodiments, the chitosan powder is at least 85%, at least 90% or at least 95% deacetylated. In some embodiments the composition comprises from about 0.5% to 5% by weight of chitosan powder.

In further embodiments, the carrier comprises water or buffered water. In an embodiment, the chitosan is dispersed in water prior to the addition of a gelling catalyst. The addition of water allows the chitosan to crosslink polymer chains upon addition of a gelling catalyst. In some embodiments, the composition comprises from 25-50% water.

Gelling of chitosan powder can be accomplished by chemical or physical means. Thus, in some embodiments the carrier further comprises a gelling catalyst. However, several known methods for gelling chitosan powder are toxic and are not suitable for biomedical uses. In certain embodiments, the gelling catalyst is non-toxic and is suitable for biomedical uses. In some instances, the gelling catalyst is a weak acid, for example, a sugar acid. Sugar acids are monosaccharides with a carboxyl group at one or both ends of its chain. Gelling catalysts suitable for gelling chitosan powder include, but are not limited to lactic acid, acetic acid, and glycolic acid. In some embodiments, the composition comprises from about 0.5% to 5% by weight of the gelling catalyst.

In some embodiments, the carrier further comprises one or more solvents. In certain embodiments, the solvent is a non-aqueous solvent. In some instances, the solvent is also a humectant, or a substance with the ability to draw moisture from the surrounding environment. In some instances, the solvent will also function as a gel plasticizing agent, transdermal vehicle, and moisturizing agent. In some embodiments, the solvent is glycerol. In other embodiments, the solvent is propylene glycol, butylene glycol, or sorbitol. In some embodiments, the composition comprises from about 0.5% to 5% by weight of the solvent.

Analgesic Active Ingredients

In one embodiment, the topical compositions provided herein comprises a therapeutically effective amount of one or more analgesic active ingredients. Analgesic agents are a class of drugs capable of relieving pain and may be classified based on their mechanism of action. Examples of analgesic agents include NSAIDs, cannabis, acetaminophen, and opioids. Additionally, other therapeutics may be classified in a separate class of chemicals but have analgesic properties. For example, certain muscle relaxers have analgesic properties.

In some embodiments, the topical composition comprises an analgesic blend of active ingredients. In some embodiments, the composition comprises at least one, two, three, four, five, six, seven, eight, nine, or ten analgesic active ingredients. In some embodiments, the composition optionally comprises additional therapeutics. In some embodiments, the additional therapeutics include muscle relaxers, anti-histamines, and anti-inflammatory agents. In certain instances, the composition optionally comprises at least one, two, three, four, five, six, seven, eight, nine, or ten additional therapeutics.

In some embodiments, the analgesic active ingredient is a nonsteroidal anti-inflammatory drug (NSAID). NSAIDs are large class of drugs that are capable of reducing pain and inflammation by inhibiting of cyclooxygenase enzymes (COX-1 or COX-2). Burian and Geisslinger, Pharmacology & Therapeutics (2005) 107: 139-154. Generally, NSAIDs are classified into two groups: non-selective and COX-2 selective. Non-selective NSAIDs are able to inhibit both COX-1 and COX-2 activity. For example NSAIDs include, acetylsalicylic acid (aspirin), methyl salicylate, sodium salicylate, trolamine salicylate, ibuprofen, flurbiprofen, naproxen, fenoprofen, pirprofen, carprofen, oxaprozin, tiaprofenic acid, diclofenac, diflunisal, etodolac, flufenamic acid, indomethacin, ketorolac, meclofenamate, mefenamic acid, nabumetone, oxyphenbutazone, phenylbutazone, piroxicam, meloxicam, salsalate, sulindac, tenoxicam, tolmetin and celecoxib. In some embodiments, the NSAID may be a non-selective NSAID. In some embodiments, the the NSAID may be a COX-2 selective NSAID.

In some embodiments, the composition comprises 2-(4-isobutylphenyl)propanoic acid (ibuprofen). In some instances, the composition comprises less than about 5%, 2%, or 1% by weight of ibuprofen.

In certain instances, the composition comprises one or more additional NDAIDs, In some embodiments, the composition comprises at least one, two, three, four five, six, seven, eight, nine or, ten NSAIDs in addition to ibuprofen. In another embodiment, the composition comprises ibuprofen and acetylsalicylic acid (aspirin). Thus, ibuprofen and aspirin can be used in combination in a topical formulation as an effective analgesic and/or anti-inflammatory agent. In some embodiments, the composition comprises from about 1-5 grams of ibuprofen per 100 grams of total composition and from about 1-5 grams of aspirin per 100 grams of total composition. In some instances, the ratio of ibuprofen to aspirin is about 4:1, 3:1, 2:1, 1:1, 1:2, 1:3, or 1:4. In some embodiments, the composition comprises less than about 5%, 2%, or 1% by weight of ibuprofen and less than about 5%, 2%, or 1% by weight of aspirin.

In some embodiments, the composition comprises one or more salicylates. For example, in some embodiments, the composition comprises aspirin, methyl salicylate, and trolamine salicylate. In certain embodiments, the composition comprises less than about 7%, 5%, 2%, or 1% by weight of each salicylate. Thus, in some embodiments the composition comprises less than about 21%, 15%, 5%, or 3% by weight of total salicylate compounds.

In some embodiments, the composition further comprises a muscle relaxant, optionally cyclobenzaprine. Or, other muscle relaxants may be used. Other muscle relaxants suitable for use with the compositions and methods described herein include, but are not limited to quinine, chlorzoxazone, and carisprodol. Muscle relaxants (e.g., cyclobenzaprine) are capable of topical application; however, their transdermal penetration is dependent upon the carrier. In some embodiments, the composition comprises from about 0.001 to about 0.01% cyclobenzaprine by weight. In some embodiments, the composition comprises 0.005% cyclobenzaprine by weight.

In still further embodiments, the disclosed composition comprises cannabidiol (CBD). CBD is a chemical present in the Cannabis sativa plant and is considered to be a non-psychoactive phytocannabinoid. CBD has been studied for its efficacy in treating several conditions, including pain and inflammation. Due to the low oral bioavailability of CBD, alternative routes of delivery are needed. In some embodiments, the disclosed topical composition or application thereof further comprises CBD. In further embodiments, the compositions may comprise between 0.001 mg and 5 grams, or 0.01 mg and 4 grams, or 0.1 mg and 2 grams of CBD, or 1 mg and 3 grams, or 10 mg and 2 grams. or 100 mg and 2 grams of CBD per 100 grams of total composition. In certain embodiments, the composition comprises from about 0.2-1% by weight of CBD.

In further embodiments, the composition comprises dimethyl sulfoxide (DMSO), an organic form of sulphur. DMSO is currently used for the treatment of inflammatory conditions and cancer and has been demonstrated to be effective a topical agent. DMSO is able to both penetrate skin and aid dermal penetration of other therapeutics. Brien et al., Osteoarthritis and Cartilage (2008) 16:1277-1288. Thus, in some embodiments, DMSO functions as an analgesic, a solvent, and a facilitator of therapeutic transdermal delivery. In some embodiments, the topical composition comprises about 1-10%, about 2-5%, or about 2.5% DMSO by weight.

In some embodiments the therapeutic agents, including the analgesic active ingredients are in powdered form. Thus, in some embodiments, the analgesic active ingredients are dissolved to form an analgesic active ingredient blend solution. In some embodiments, the powdered therapeutic agents may be dissolved using a primary non-aqueous solvent. In addition to being capable of dissolving the powdered therapeutic agents, the solvent may have other charcteristics. Thus, in some embodiments, the solvent is suitable for biomedical uses, including skin contact, has low toxicity, and serves as a quick-drying agent. In some embodiments, the solvent is a lower alcohol. Lower alcohols are colorless liquids at normal temperatures and include ethanol, propanol, and isopropanol. In some embodiments, the solvent is ethanol. In other embodiments, the solvent is a liniment base. Any liniment base known in the art is suitable for use as a solvent. Examples of such liniment bases include mixtures composed of 10-70 parts by weight of an alcohol such as but not limited to a monohydric alcohol (e.g., ethanol, propanol, isopropyl alcohol), a polyhydric alcohol (e.g., polyethylene glycol, propylene glycol, butylene glycol), or the like), up to 55 parts by weight of water, up to 60 parts by weight of a fatty acid ester (e.g., an ester of adipic acid, sebacic acid, or myristic acid) and up to 10 parts by weight of a surfactant (e.g., polyoxyethylene alkyl ether). If necessary, for example, neutralizing agents (for pH adjustment), tackifiers (e.g., methyl cellulose, carboxyvinyl polymer, or hydroxypropyl cellulose), rash-preventing agents, and other additives (e.g., salicylic acid, methyl salicylate, glycol salicylate, 1-menthol, camphor; peppermint oil, capsicum, extract, nonylic vanillylamide, crotamiton, AzoneR, propylene carbonate, or diisopropyl adipate) may also be added in the liniment. For example, 0.1-10 parts by weight of the non-steroidal anti-inflammatory analgesic, 0.001-0.2 part by weight of an alkyl ester of gallic acid and 0.01-10 parts by weight of a phenolic radical scavenger may be added to a liniment base to obtain a liniment (i.e., liquid or lotion for applying to the body to relieve pain).

In some embodiments, the composition is a topical formulation. In certain embodiments, the topical formulation is a gel, cream, ointment, foam, powder, emulsion, lotion, a spray, or any other topical formulation generally known in the art. In certain embodiments, the topical formulation is a clear gel.

In some embodiments the composition is capable of being stored for at least 6 months, 1 year, 2 years, or 5 years at 4-40° C.

Methods of Treatment

In other embodiments, provided are methods for preventing and/or treating or reducing pain and/or inflammation in a subject. The subject may be any animal. In an embodiment, the subject is a human. In some embodiments, the subject has inflammation and/or pain. In certain embodiments, the method comprises topically administering to the subject a composition comprising a therapeutically effective amount of any one of the compositions described herein.

In some embodiments, the method for preventing and/or treating or reducing pain and/or inflammation in a subject comprises topically administering to the subject any one of the compositions described herein to the skin or mucosal surtace. Transdermal delivery can improve the efficiency and therapeutic bioavailability, in part, by avoiding the first-pass metabolism that impacts oral drug delivery.

Transdermal delivery of therapeutics primarily occurs via stratum comeum, which consists of dead, keratinized epidermal cells. Delivery of drugs across the epidermal cells can be challenging. Thus, in certain embodiments, the size and properties of the therapeutics as well as the properties of the delivery system determine whether a therapeutic can be delivered transdermally.

In some embodiments of the method, the one or more active analgesic ingredients are delivered in a time-release manner. In some embodiments, application of one or more of the compositions described herein results in the formation of a film or bioadhesive on the surface of the skin. The unique chemical and physical properties of the chitosan-based gel carrier result in the formation of a thin, patch-like film when the gel is applied to skin. The ability of the chitosan-based gel formulation to form a film is significant in that it overcomes several of the disadvantages and limitation of other delivery systems. For example, lotions, ointments, and creams are easily wiped off and require frequent re-application. The use of patches and bandages is also not ideal in that they are prone to causing skin irritation and are difficult to fit over certain areas of the body. The film-forming delivery system described herein allows for the carrier to remain in contact with the skin for prolonged periods of time and deliver the therapeutics in a time-release manner.

Thus, in some embodiments, the chitosan gel carrier allows for increased therapeutic delivery efficiency. Due to the increased efficiency in therapeutic delivery, lower concentrations of therapeutic agents may be used in the compositions and methods disclosed herein. For example, the method comprises topically administering a composition comprising less than 5%, 2%, or 1% ibuprofen to the surface of the skin.

The ability of the chitosan-based gel formulation to stably carry multiple therapeutic agents in the same formulation provides a significant advantage in that these therapeutics are able to be delivered together to create a synergistic effect. In some embodiments, the method comprises topically administering a composition comprising ibuprofen and at least one, two, three, four, five, six, seven, eight, nine, or ten additional NSAIDs, and optionally at least one, two, three, four, five, six, seven, eight, nine, or ten additional therapeutic agents. For example in some embodiments, the method comprises topically administering ibuprofen and aspirin and optionally administering CBD and cyclobenzaprine to a treatment site. Topical sites treatable through the use of the compositions described herein include, but are not limited to forehead, arms, legs, chest, back, neck, hands, feet.

In some embodiments, the composition is applied preemptively, retroactively or both preemptively and retroactively. Thus, in some embodiments, the composition is used proactively to prevent pain and/or inflammation. In certain embodiments, the composition is used retroactively to treat pain and/or inflammation.

In some embodiments, the methods and compositions described herein may be used for the treatment of acute pain. In other embodiments, the methods and compositions described herein may be used for the treatment of chronic pain. In certain instances, the methods and compositions described herein may be used for treatment of pain, including but not limited to neck pain, back pain, joint pain, migraine pain, and muscular pain. In some embodiments, the methods and compositions described herein may be used to treat inflammatory diseases such as arthritis.

In some embodiments, the topical composition is administered using a patch or bandage. Because the chitosan gel-based delivery is able to form a protective skin upon contact with the skin, it is not necessary to apply the topical analgesic composition to the skin on a separate carrier (e.g., a bandage or dressing). In some embodiments, the composition is applied directly to the skin.

The composition may in certain embodiments be provided at least 1, 2, 3, 4, or 5 times a day for at least 1, 2, 3, 4, 5, 6, 7, 8, 9, or 10 days. Or, longer periods of application may be used.

### Examples

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter.

The following examples describe methods for the preparations and use of a topical analgesic gel.

### Example 1. Preparation of a Topical Analgesic Gel

### Materials

The following materials were used in the examples and methods below:
1. Chitosan Powder, ≥95% deacetylation
2. Lactic Acid, 85%, CAS #50-21-5
3. Glycerol, 99%+, CAS #56-81-5
4. DiH2O, CAS #7732-18-5
5. Ethanol, 200 proof, CAS #64-17-5
6. Dimethyl Sulfoxide, “DMSO”, 99%, CAS #67-68-5
7. Ibuprofen, USP grade, CAS #15687-27-1
8. Methyl Salicylate, 99%, CAS #119-36-8

A chitosan gel was prepared by combining 32.5 g of deacetylated chitosan powder, 100 g of glycerol, and 835 g of deionized (DiH₂O) into a bottle. The solution is then mixed until the chitosan powder is dispersed. Then 32.5 g of lactic acid was added to the solution while slowly stirring until a thick, clear gel of 3.25% chitosan was formed.

Next, the analgesic active ingredient blend solution was prepared. In a first formulation (TABLE 1), 20 g of ibuprofen powder, 20 g of methyl salicylate and 460 mL of ethanol were combined and the solution was shaken to dissolve the powder. Next 450 g of the chitosan gel was added to the solution and shaken/mixed until the solution turned completely clear. Lastly, 50 g of DMSO was added to the solution.

Using the method described above, various formulations were prepared according to the specifications provided in Tables 2-24.

TABLE 1
Formulation # 1 NBC-1020

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 464.990 | 46.500 |
| Analgesic | DMSO | 25.000 | 2.500 |
| Blend Active | Methyl Salicylate | 20.000 | 2.000 |
| Ingredients | Aspirin | 20.000 | 2.000 |
| | Ibuprofen | 20.000 | 2.000 |

TABLE 2
Formulation # 2: NBC-2020

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 459.990 | 46.000 |
| Analgesic | DMSO | 25.000 | 2.500 |
| Blend Active | Methyl Salicylate | 20.000 | 2.000 |
| Ingredients | Aspirin | 20.000 | 2.000 |
| | Ibuprofen | 20.000 | 2.000 |
| | CBD Extract | 5.000 | 0.500 |

TABLE 3
Formulation # 3: NBC-3020

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 464.940 | 46.495 |
| Analgesic | DMSO | 25.000 | 2.500 |
| Blend Active | Methyl Salicylate | 20.000 | 2.000 |
| Ingredients | Aspirin | 20.000 | 2.000 |
| | Ibuprofen | 20.000 | 2.000 |
| | Cyclobenzoprine | 0.050 | 0.005 |

TABLE 4
Formulation # 4: NBC-4020

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 459.940 | 45.995 |
| Analgesic | DMSO | 25.000 | 2.500 |
| Blend Active | Methyl Salicylate | 20.000 | 2.000 |
| Ingredients | Aspirin | 20.000 | 2.000 |
| | Ibuprofen | 20.000 | 2.000 |
| | CBD Extract | 5.000 | 0.500 |
| | Cyclobenzoprine | 0.050 | 0.005 |

TABLE 5
Formulation # 5: NBC-1021

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 434.990 | 43.500 |
| Analgesic | DMSO | 25.000 | 2.500 |
| Blend Active | Methyl Salicylate | 20.000 | 2.000 |
| Ingredients | Aspirin | 50.000 | 5.000 |
| | Ibuprofen | 20.000 | 2.000 |

TABLE 6
Formulation # 6: NBC-2021

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 429.990 | 43.000 |
| Analgesic | DMSO | 25.000 | 2.500 |
| Blend Active | Methyl Salicylate | 20.000 | 2.000 |
| Ingredients | Aspirin | 50.000 | 5.000 |
| | Ibuprofen | 20.000 | 2.000 |
| | CBD Extract | 5.000 | 0.500 |

TABLE 7
Formulation # 7: NBC-3021

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 434.940 | 43.495 |
| Analgesic | DMSO | 25.000 | 2.500 |
| Blend Active | Methyl Salicylate | 20.000 | 2.000 |
| Ingredients | Aspirin | 50.000 | 5.000 |
| | Ibuprofen | 20.000 | 2.000 |
| | Cyclobenzoprine | 0.050 | 0.005 |

TABLE 8
Formulation # 8: NBC-4021

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 429.940 | 42.995 |
| Analgesic | DMSO | 25.000 | 2.500 |
| Blend Active | Methyl Salicylate | 20.000 | 2.000 |
| Ingredients | Aspirin | 50.000 | 5.000 |
| | Ibuprofen | 20.000 | 2.000 |
| | CBD Extract | 5.000 | 0.500 |
| | Cyclobenzoprine | 0.050 | 0.005 |

TABLE 9
Formulation # 9: NBC-1022

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 464.990 | 46.500 |
| Analgesic | DMSO | 25.000 | 2.500 |
| Blend Active | Methyl Salicylate | 20.000 | 2.000 |
| Ingredients | Trolamine Salicylate | 20.000 | 2.000 |
| | Ibuprofen | 20.000 | 2.000 |

TABLE 10
Formulation # 10: NBC-2022

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 459.990 | 46.000 |
| Analgesic | DMSO | 25.000 | 2.500 |
| Blend Active | Methyl Salicylate | 20.000 | 2.000 |
| Ingredients | Trolamine Salicylate | 20.000 | 2.000 |
| | Ibuprofen | 20.000 | 2.000 |
| | CBD Extract | 5.000 | 0.500 |

TABLE 11
Formulation # 11: NBC-3022

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 464.940 | 46.495 |
| Analgesic | DMSO | 25.000 | 2.500 |
| Blend Active | Methyl Salicylate | 20.000 | 2.000 |
| Ingredients | Trolamine Salicylate | 20.000 | 2.000 |
| | Ibuprofen | 20.000 | 2.000 |
| | Cyclobenzoprine | 0.050 | 0.005 |

TABLE 12
Formulation # 12: NBC-4022

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 459.940 | 45.995 |
| Analgesic | DMSO | 25.000 | 2.500 |
| Blend Active | Methyl Salicylate | 20.000 | 2.000 |
| Ingredients | Trolamine Salicylate | 20.000 | 2.000 |
| | Ibuprofen | 20.000 | 2.000 |
| | CBD Extract | 5.000 | 0.500 |
| | Cyclobenzoprine | 0.050 | 0.005 |

TABLE 13
Formulation # 13: NBC-1023

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 434.990 | 43.500 |
| Analgesic | DMSO | 25.000 | 2.500 |
| Blend Active | Methyl Salicylate | 20.000 | 2.000 |
| Ingredients | Trolamine Salicylate | 50.000 | 5.000 |
| | Ibuprofen | 20.000 | 2.000 |

TABLE 14
Formulation # 14; NBC-2023

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 429.990 | 43.000 |
| Analgesic | DMSO | 25.000 | 2.500 |
| Blend Active | Methyl Salicylate | 20.000 | 2.000 |
| Ingredients | Trolamine Salicylate | 50.000 | 5.000 |
| | Ibuprofen | 20.000 | 2.000 |
| | CBD Extract | 5.000 | 0.500 |

TABLE 15
Formulation # 15: NBC-3023

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 434.940 | 43.495 |
| Analgesic | DMSO | 25.000 | 2.500 |
| Blend Active | Methyl Salicylate | 20.000 | 2.000 |
| Ingredients | Trolamine Salicylate | 50.000 | 5.000 |
| | Ibuprofen | 20.000 | 2.000 |
| | Cyclobenzoprine | 0.050 | 0.005 |

TABLE 16
Formulation # 16: NBC-4023

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DIH2O | 375.750 | 37.575 |
| | Ethanol | 429.940 | 42.995 |
| Analgesic | DMSO | 25.000 | 2.500 |
| Blend Active | Methyl Salicylate | 20.000 | 2.000 |
| Ingredients | Trolamine Salicylate | 50.000 | 5.000 |
| | Ibuprofen | 20.000 | 2.000 |
| | CBD Extract | 5.000 | 0.500 |
| | Cyclobenzoprine | 0.050 | 0.005 |

TABLE 17
Formulation # 17: NBC-1015

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 494.990 | 49.500 |
| Analgesic | DMSO | 25.000 | 2.500 |
| Blend Active | Methyl Salicylate | 20.000 | 2.000 |
| Ingredients | Diclofenac | 10.000 | 1.000 |

TABLE 18
Formulation # 18: NBC-2015

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 489.990 | 49.000 |
| Analgesic | DMSO | 25.000 | 2.500 |
| Blend Active | Methyl Salicylate | 20.000 | 2.000 |
| Ingredients | Diclofenac | 10.000 | 1.000 |
| | CBD Extract | 5.000 | 0.500 |

TABLE 19
Formulation # 19: NBC-3015

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 474.940 | 47.495 |
| Analgesic | DMSO | 25.000 | 2.500 |
| Blend Active | Methyl Salicylate | 20.000 | 2.000 |
| Ingredients | Diclofenac | 10.000 | 1.000 |
| | Ibuprofen | 20.000 | 2.000 |
| | Cyclobenzoprine | 0.050 | 0.005 |

TABLE 20
Formulation # 20: NBC-4015

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 489.940 | 48.995 |
| Analgesic | DMSO | 25.000 | 2.500 |
| Blend Active | Methyl Salicylate | 20.000 | 2.000 |
| Ingredients | Diclofenac | 10.000 | 1.000 |
| | CBD Extract | 5.000 | 0.500 |
| | Cyclobenzoprine | 0.050 | 0.005 |

TABLE 21
Formulation # 21: NBC-1024

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 444.990 | 44.500 |
| Analgesic | DMSO | 25.000 | 2.500 |
| Blend Active | Methyl Salicylate | 20.000 | 2.000 |
| Ingredients | Aspirin | 50.000 | 5.000 |
| | Diclofenac | 10.000 | 1.000 |

TABLE 22
Formulation # 22: NBC-2024

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 439.990 | 44.000 |
| Analgesic | DMSO | 25.000 | 2.500 |
| Blend Active | Methyl Salicylate | 20.000 | 2.000 |
| Ingredients | Aspirin | 50.000 | 5.000 |
| | Diclofenac | 10.000 | 1.000 |
| | CBD Extract | 5.000 | 0.500 |

TABLE 23
Formulation # 23: NBC-3024

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 464.940 | 46.495 |
| Analgesic | DMSO | 25.000 | 2.500 |
| Blend Active | Methyl Salicylate | 20.000 | 2.000 |
| Ingredients | Aspirin | 20.000 | 2.000 |
| | Ibuprofen | 20.000 | 2.000 |
| | Cyclobenzoprine | 0.050 | 0.005 |

TABLE 24
Formulation # 24: NBC-4024

| | Materials: | g/L | % |
|---|---|---|---|
| Gel Base: | Chitosan Powder | 14.630 | 1.463 |
| | Lactic Acid | 14.630 | 1.463 |
| | Glycerol | 45.000 | 4.500 |
| | DiH2O | 375.750 | 37.575 |
| | Ethanol | 459.940 | 45.995 |
| Analgesic | DMSO | 25.000 | 2.500 |
| Blend Active | Methyl Salicylate | 20.000 | 2.000 |
| Ingredients | Aspirin | 20.000 | 2.000 |
| | Ibuprofen | 20.000 | 2.000 |
| | CBD Extract | 5.000 | 0.500 |
| | Cyclobenzoprine | 0.050 | 0.005 |

### Example 2. Treatment of Athletes with Topical Analgesic Gel

Briefly, twenty male tennis player from 30-60 years of age experiencing pain in various areas of their body (e.g., knee, lower back, shoulder, elbow, and forearm) were instructed to use a 2% ibuprofen, 5% aspirin, 2% methyl salicylate, and 5% DMSO formulation of the topical analgesic gel during a 2.5 month long tennis season in replacement of their normal pre- and post-match oral pain relievers (e.g., ibuprofen, naproxen, and aspirin). All athletes were instructed not to use any oral or topical pain relief medications concurrently with the topical analgesic gel.

100% of athletes indicated they achieved equal or better pain relief using the topical gel on localized body parts where they regularly experienced pain that they normally treated with oral pain relievers. Three athletes who regularly used pre-match oral pain relievers for knee soreness found that they were able to achieve equal-or-better pain relief during and after their matches following application of the topical analgesic gel to each knee 5-10 minutes before the match began (real-time dosing).

### Illustrations of Suitable Compositions and Methods

Illustration A1 is a composition formulated for topical application comprising: a therapeutically effective amount of one or more analgesic active ingredients and a carrier gel comprising chitosan.

Illustration A2 is the composition of any preceding or subsequent illustration, wherein the carrier gel comprises chitosan powder, a gelling catalyst, and a solvent.

Illustration A3 is the composition of any preceding or subsequent illustration, wherein the composition comprises from about 0.5% to 5% by weight of chitosan powder.

Illustration A4 is the composition of any preceding or subsequent illustration, wherein the composition comprises from about 0.5% to 5% by weight of the gelling catalyst.

Illustration A5 is the composition of any preceding or subsequent illustration, wherein the composition comprises from about 0.5% to 5% by weight of the solvent.

Illustration A6 is the composition of any preceding or subsequent illustration, wherein the gelling catalyst comprises lactic acid.

Illustration A7 is the composition of any preceding or subsequent illustration, wherein the solvent comprises glycerol.

Illustration A8 is the composition of any preceding or subsequent illustration, wherein the active ingredient comprises a NSAID.

Illustration A9 is the composition of any preceding or subsequent illustration, wherein the NSAID comprises ibuprofen.

Illustration A10 is the composition of any preceding or subsequent illustration, further comprising one or more additional NSAIDs, wherein the one or more additional NSAID comprises at least one of acetylsalicylic acid (aspirin), methyl salicylate, sodium salicylate, trolamine salicylate, flurbiprofen, naproxen, fenoprofen, pirprofen, carprofen, oxaprozin, tiaprofenic acid, diclofenac, diflunisal, etodolac, flufenamic acid, indomethacin, ketorolac, meclofenamate, mefenamic acid, nabumetone, oxyphenbutazone, phenylbutazone, piroxicam, meloxicam, salsalate, sulindac, tenoxicam, tolmetin or celecoxib.

Illustration A11 is the composition of any preceding or subsequent illustration, wherein the additional NSAID is acetylsalicylic acid.

Illustration A12 is the composition of any preceding or subsequent illustration, wherein the composition comprises less than about 5% by weight of ibuprofen and less than about 5% by weight of acetylsalicylic acid.

Illustration A13 is the composition of any preceding or subsequent illustration, wherein the composition comprises less than about 2% by weight of ibuprofen and less than about 2% by weight of acetylsalicylic acid.

Illustration A14 is the composition of any preceding or subsequent illustration, wherein the composition further comprises a muscle relaxant.

Illustration A15 is the composition of any preceding or subsequent illustration, wherein the muscle relaxant is a cyclobenzaprine.

Illustration A16 is the composition of any preceding or subsequent illustration, wherein the composition comprises from about 0.0025-0.01% by weight of the cyclobenzaprine.

Illustration A17 is the composition of any preceding or subsequent illustration, wherein the composition further comprises CBD.

Illustration A18 is the composition of any preceding or subsequent illustration, wherein the composition comprises from about 0.2-1% by weight of CBD.

Illustration A19 is the composition of any preceding or subsequent illustration, wherein the composition further comprises DMSO.

Illustration A20 is the composition of any preceding or subsequent illustration, wherein the composition comprises from about 2-10% by weight of DMSO.

Illustration A21 is the composition of any preceding or subsequent illustration, wherein the solvent further comprises water or buffered water.

B1 is a method for preventing and/or treating and/or reducing pain and/or inflammation in a subject comprising topically administering to the subject any one of the compositions of illustrations A1-A21.

Illustration B2 is the method of any preceding or subsequent illustration, wherein the composition is applied to the skin or mucosal surface.

What is claimed:

1. A composition formulated for topical application comprising: a therapeutically effective amount of one or more analgesic active ingredients and a carrier gel comprising chitosan.

2. The composition of claim 1, wherein the carrier gel comprises a chitosan powder, a gelling catalyst, and a solvent.

3. The composition of any one of claims 1-2, wherein the composition comprises from about 0.5% to 5% by weight of chitosan powder.

4. The composition of any one of claims 1-2, wherein the composition comprises from about 0.5% to 5% by weight of the gelling catalyst.

5. The composition of any one of claims 1-2, wherein the composition comprises from about 0.5% to 5% by weight of the solvent.

6. The composition of any one of claims 2-5, wherein the gelling catalyst comprises lactic acid.

7. The composition of any one of claim 1-6, wherein the solvent comprises glycerol.

8. The composition of any one of claims 1-7, wherein the active ingredient comprises a NSAID.

9. The composition of claim 8, wherein the NSAID comprises ibuprofen.

10. The composition of claim 9, further comprising one or more additional NSAIDs, wherein the one or more additional NSAID comprises at least one of acetylsalicylic acid (aspirin), methyl salicylate, sodium salicylate, trolamine salicylate, flurbiprofen, naproxen, fenoprofen, pirprofen, carprofen, oxaprozin, tiaprofenic acid, diclofenac, diflunisal, etodolac, flufenamic acid, indomethacin, ketorolac, meclofenamate, mefenamic acid, nabumetone, oxyphenbutazone, phenylbutazone, piroxicam, meloxicam, salsalate, sulindac, tenoxicam, tolmetin or celecoxib.

11. The composition of claim 10, wherein the additional NSAID is acetylsalicylic acid.

12. The composition of claim 11, wherein the composition comprises less than about 5% by weight of ibuprofen and less than about 5% by weight of acetylsalicylic acid.

13. The composition of claim 11, wherein the composition comprises less than about 2% by weight of ibuprofen and less than about 2% by weight of acetylsalicylic acid.

14. The composition of any one of claims 1-13, wherein the composition further comprises a muscle relaxant.

15. The composition of claim 14, wherein the muscle relaxant is a cyclobenzaprine.

16. The composition of claim 15, wherein the composition comprises from about 0.0025-0.01% by weight of the cyclobenzaprine.

17. The composition of any one of claims 1-16, wherein the composition further comprises CBD.

18. The composition of claim 17, wherein the composition comprises from about 0.2-1% by weight of CBD.

19. The composition of any one of claims 1-18, wherein the composition further comprises DMSO.

20. The composition of claim 19, wherein the composition comprises from about 2-10% by weight of DMSO.

21. The composition of any one of claims 2-20, wherein the solvent further comprises water or buffered water.

22. A method for preventing and/or treating and/or reducing pain and/or inflammation in a subject comprising topically administering to the subject any one of the compositions of claims 1-21.

23. The method of claim 22, wherein the composition is applied to the skin or mucosal surface.
